(19) United States
(12) Patent Application Publication
(10) Pub. No.: US 2021/0004997 A1
(43) Pub. Date: Jan. 7, 2021

(54) METHOD FOR PROVIDING AN EVALUATION DATASET FROM A FIRST MEDICAL THREE-DIMENSIONAL COMPUTED TOMOGRAPHY DATASET

(71) Applicant: Siemens Healthcare GmbH, Erlangen (DE)

(72) Inventors: Michael Manhart, Furth (DE); Markus Kowarschik, Nurnberg (DE); Christian Kaethner, Forchheim (DE)

(21) Appl. No.: 16/916,761

(22) Filed: Jun. 30, 2020

(30) Foreign Application Priority Data
Jul. 3, 2019 (DE) 102019209790.1

(51) Int. Cl. G06T 11/00; G06T 7/11; G06T 5/00; G06T 3/60; G06T 7/00; A61B 6/03; A61B 6/00; G16H 50/20

(52) U.S. Cl. CPC G06T 11/008; G06T 7/11; G06T 5/002; G06T 3/60; G06T 7/0012; G06T 2207/10081; A61B 6/5264; A61B 6/5217; A61B 6/507; G16H 50/20; G06T 2207/30101; A61B 6/032

(57) ABSTRACT

A system and method for providing an evaluation dataset from a first medical three-dimensional computed tomography dataset. The method includes reconstructing the first three-dimensional computed tomography dataset from a plurality of two-dimensional X-ray projection images recorded with different acquisition geometries from an examination object by a medical X-ray device and providing an artifact-reduced image dataset. Providing includes applying a method for reducing artifacts to the first computed tomography dataset. The method further includes identifying for example hemorrhagic and/or ischemic stroke indications by applying an method for identifying stroke indications to the artifact-reduced image dataset, creating an evaluation dataset by applying an method for evaluating a manifestation of the identified stroke indications to the artifact-reduced image dataset, and providing the evaluation dataset.

### CROSS REFERENCE TO RELATED APPLICATIONS

This application claims the benefit of DE 102019209790.1 filed on Jul. 3, 2019 which is hereby incorporated by reference in its entirety.

### FIELD

Embodiments relate to a method for providing an evaluation dataset from a first medical three-dimensional computed tomography dataset, a medical X-ray device, and a computer program product.

### BACKGROUND

The time between the admission of a stroke patient to hospital until the targeted treatment of the stroke is of decisive importance for a good treatment outcome. It is known that methods for stroke examination frequently require a plurality of imaging and image evaluation steps in temporal succession. The brain of the stroke patient is examined by a computed tomography system and/or a medical X-ray device and/or a magnetic resonance system. Methods for stroke examination often precede the treatment for the stroke.

In view of the diversity of the different types of stroke and the respective manifestations thereof, a lot of time is lost during the stroke examination. An operator frequently has to carry out the individual method steps. The stroke examination, for example the course of the method, is frequently error prone and time consuming. Assistance for the operator, for example a physician and/or surgeon, during the performance of a stroke examination, for example by technical and objective devices, would be helpful during the performance of a stroke examination.

### BRIEF SUMMARY AND DESCRIPTION

The scope of the present disclosure is defined solely by the appended claims and is not affected to any degree by the statements within this summary. The present embodiments may obviate one or more of the drawbacks or limitations in the related art.

Embodiments provide a reliable and time-efficient method for improving stroke examinations.

Embodiments provide where a first three-dimensional computed tomography dataset is reconstructed from a plurality of two-dimensional X-ray projection images recorded with different acquisition geometries from an examination object by a medical X-ray device. In addition, an artifact-reduced image dataset is provided. The provision includes applying a method for reducing artifacts to the first computed tomography dataset. Further, hemorrhagic and/or ischemic stroke indications are identified by applying a method for identifying stroke indications to the artifact-reduced image dataset. Moreover, an evaluation dataset is created by applying a method for evaluating a manifestation of the identified stroke indications to the artifact-reduced image dataset. In addition, the evaluation dataset is provided.

The examination object may, for example, be an animal patient and/or a human patient.

The medical X-ray device may be configured as a rotationally movable, for example C-arm, X-ray device and/or as a computed tomography system (CT). The medical X-ray device may be configured to record the plurality of two-dimensional X-ray projection images with different acquisition geometries from one another, for example about a common axis of rotation, from the examination object. The common axis of rotation may correspond to a longitudinal axis of the examination object and/or a patient positioning facility on which the examination object is arranged.

The acquisition geometries may include an X-ray projection direction, for example relative to the examination object and/or a patient positioning facility, and/or an X-ray geometry, for example a cone beam geometry and/or fan beam geometry. As a result, redundancies, for example intersections and/or multiple scans of mappings of regions of the examination object, may occur during the recording of the plurality of two-dimensional X-ray projection images. This may provide the reconstruction of the first three-dimensional computed tomography dataset from the plurality of two-dimensional X-ray projection images, for example by an inverse Radon transform. The different acquisition geometries are configured such that a spatial region of a brain of the examination object is at least partially mapped by the plurality of two-dimensional X-ray projection images.

In addition, the method may include recording the plurality of two-dimensional X-ray projection images for reconstructing the first three-dimensional computed tomography dataset by the medical X-ray device. Further, the method may include reception of the plurality of two-dimensional X-ray projection images for reconstructing the first three-dimensional computed tomography dataset. The reception of the plurality of two-dimensional X-ray projection images for reconstructing the first three-dimensional computed tomography dataset may include acquisition and/or reading of a computer-readable storage medium and/or reception from a data storage unit, for example a database.

The application of the method for reducing artifacts to the first computed tomography dataset provides all disruptive image portions, that may, for example, mask and/or fog image portions, to be removed. Artifacts may for example include disruptive image portions in the first three-dimensional computed tomography dataset, for example noise and/or metal artifacts and/or motion artifacts. Further, the diagnostic image portions are retained in the artifact-reduced image dataset provided. In addition, the artifact-reduced image dataset is three-dimensional and includes the same spatial resolution as the first computed tomography dataset.

The provision of the artifact-reduced image dataset may for example include storage on a computer-readable storage medium and/or displaying on a depiction unit and/or transfer to a processing unit.

In addition, stroke indications may include all the features of the artifact-reduced image dataset, that for example are directly and/or implicitly characteristic of a stroke. It is, for example, possible to differentiate between hemorrhagic and/or ischemic stroke indications. Hemorrhagic stroke indications may, for example, include internal bleeding, for example in tissue of the examination object that is adjacent to a vessel. Hemorrhagic stroke indications may be identified using an image contrast difference in the artifact-reduced image dataset. Further, a contrast medium and a contrast resulting therefrom may be taken into account for depicting hemorrhagic stroke indications.

Ischemic stroke indications may, for example, include a thrombus and/or a necrotic area and/or a penumbral area.

The identification of the, for example hemorrhagic and/or ischemic, stroke indications may include the determination of a spatial extent and/or a spatial course and/or a position and/or an alignment and/or a morphological condition and/or a density of the respective stroke symptom in the artifact-reduced image dataset.

Further, the method applied to the artifact-reduced image dataset to create the evaluation dataset is configured to evaluate a manifestation of the identified stroke indications. The evaluation of the manifestation of the identified stroke indications may, for example, include an evaluation according to the “Alberta Stroke Program Early CT Score” (ASPECTS). For example, the evaluation of the manifestation of the identified stroke indications may include a comparison between two hemispheres of the brain of the examination object mapped in the artifact-reduced image dataset. The evaluation of the identified stroke indications may furthermore include an evaluation of the spatial extent and/or the spatial course and/or the position and/or the alignment and/or the morphological condition and/or the density of the respective stroke symptom in the artifact-reduced image dataset. The evaluation of the manifestation of the identified stroke indications may include the assignment of a value, for example a dimension, and/or a value tuple to at least one, for example all, identified stroke indications.

The evaluation dataset created by applying the method for evaluating the manifestation of the identified stroke indications to the artifact-reduced image dataset may include information, for example assigned values, for example dimensions, and/or value tuples, for the evaluation of the identified stroke indications. Further, the evaluation dataset may for example be used to classify the identified stroke indications. For example, the evaluation of the manifestation of the identified stroke indications may be used to differentiate between a stroke, for example causally, on the middle cerebral artery (MCA) and/or the anterior cerebral artery (ACA) and/or the posterior cerebral artery (PCA) and/or the internal carotid artery (ICA) and/or the basilar artery (BA) and/or the cerebellar arteries and/or the vertebral artery.

The provision of the evaluation dataset may for example include storage on a computer-readable storage medium and/or displaying on a depiction unit and/or transfer to a processing unit. The evaluation dataset provided may be useful for example for improved diagnostic assistance for an operator, for example a physician.

In a further embodiment, the method may furthermore include the creation of a three-dimensional thrombus image from the artifact-reduced image dataset or the evaluation dataset. A thrombus may be identified as a stroke symptom. Further, the creation of the three-dimensional thrombus image may include segmentation of the thrombus from the artifact-reduced image dataset and/or the evaluation dataset. In addition, the thrombus image may be provided.

A thrombus may for example be formed by a blood clot in a blood vessel of the examination object, for example in the brain. The evaluation dataset is configured as two-dimensional and/or three-dimensional. Information for evaluating the manifestation of the thrombus as an identified stroke symptom provides the thrombus to be used for segmentation. Further, as an identified stroke symptom, the thrombus may be segmented from the artifact-reduced image dataset. The segmentation may also take place taking account of the evaluation dataset. For example, the evaluation dataset, for example the values and/or value tuples contained therein, may be used for better differentiation of the thrombus from the surrounding tissue.

Further, the segmentation of the thrombus may for example take place based on values of voxels of the artifact-reduced image dataset and/or the evaluation dataset. For example, at least one threshold value may be specified. All voxels with a value below and/or above the at least one specified threshold value may be segmented from the artifact-reduced image dataset and/or the evaluation dataset as a thrombus voxel. Further, a value interval may be specified. All voxels with a value within and/or outside the specified value interval may be segmented from the artifact-reduced image dataset and/or the evaluation dataset as a thrombus voxel. A thrombus voxel describes a voxel that at least partially spatially maps a thrombus.

The thrombus image created from the artifact-reduced image dataset and/or the evaluation dataset includes mapping of the segmented thrombus. Other image portions, which for example correspond to tissue types of the examination object different from the thrombus, are not contained in the thrombus image. Further, the three-dimensional thrombus image may include a plurality of voxels. A voxel may be formed by a three-dimensional image point, for example in the three-dimensional thrombus image.

The provision of the three-dimensional thrombus image may for example include storage on a computer-readable storage medium and/or displaying on a depiction unit and/or transfer to a processing unit.

In a further embodiment of the method, the method for reducing artifacts may include registering the two-dimensional X-ray projection images to one another and/or applying a filter for noise reduction and/or motion correction.

The method for reducing artifacts may include registering the plurality of two-dimensional X-ray images, for example to one another and/or with respect to a common reference point, and/or a noise reduction, for example applying a noise filter, and/or motion correction.

Further, the method for reducing artifacts may include a method for noise reduction in computed tomography data according to the as yet unpublished German patent application 102019202878.0 hereby incorporated in its entirely by reference.

Motion correction of the plurality of two-dimensional X-ray projection images may, for example, be enabled by an external sensor signal, for example a motion sensor and/or a breathing sensor and/or a heart sensor, and/or using a comparison between the mapping of the examination object in the plurality of two-dimensional X-ray projection images.

In a further embodiment of the method, the method for reducing artifacts may include the following steps: creating a three-dimensional auxiliary dataset by undersampling the first three-dimensional computed tomography dataset; determining in each case a slice along two spatial directions of the three-dimensional auxiliary dataset, wherein each of the two slices in each case includes a predetermined column and, in each case, at least one line; determining at least one rotation parameter and/or at least one translation parameter for reducing a line-by-line deviation with respect to the in each case predetermined column in each of the two slices; and correcting the first three-dimensional computed tomography dataset by applying the at least one rotation parameter and/or the at least one translation parameter.

The undersampling of the first three-dimensional computed tomography dataset to create the three-dimensional auxiliary dataset may, for example, take place by a transfer function and/or a window function. This, for example, provides significant regions of the first three-dimensional computed tomography dataset that are particularly suitable for the determination of the at least one rotation parameter and/or the at least one translation parameter to be subsampled to a lower degree than, for example, less suitable regions. For example, the undersampling may take place based on a content-based weighting of at least one region of the first three-dimensional computed tomography dataset. For example, edges and/or corners and/or high-contrast objects, for example bones, may be subsampled to a lesser degree.

Further, the undersampling of the first three-dimensional computed tomography dataset may take place uniformly along at least one, for example axial and/or coronal and/or sagittal, spatial direction. For example, a predetermined number of voxels may be skipped during the undersampling and hence during the creation of the three-dimensional auxiliary dataset.

The three-dimensional auxiliary dataset includes a lower number of voxels than the first three-dimensional computed tomography dataset.

The determination of the at least one rotation parameter and/or the at least one translation parameter may for example take place iteratively. In each case a slice may be determined and/or specified along two spatial directions. The determination of the at least one rotation parameter and/or the at least one translation parameter may include an iterative correction of the three-dimensional auxiliary dataset by applying the in each case most recently determined at least one rotation parameter and/or at least one translation parameter. The correction may include at least one rotation and/or translation of the three-dimensional auxiliary dataset with respect to the respective predetermined and/or specified slice. Further, the correction of the three-dimensional auxiliary dataset may include re-rasterization.

The determination of the at least one rotation parameter and/or the at least one translation parameter in the next iteration step may in each case take place in the same predetermined and/or specified slice along two spatial directions that are for example the same as in the previous iteration step. The respective slice may in each case after an iteration step have a different content, for example due to the correction of the three-dimensional auxiliary dataset.

The line-by-line deviation with respect to the predetermined, for example central, column may be ascertained by the values of the voxels of the respective line on the first side, for example on the left, or on the second side, for example on the right, of the predetermined column within the same slice. For example, the values of the voxels of the respective line on the first side of the predetermined column may be used to form a first value, for example by summation and/or multiplication and/or division. Further, the values of the voxels of the same line on the second side of the predetermined column may be used to form a second value, for example by summation and/or multiplication and/or division. The deviation for the respective line may be ascertained by a comparison of the first and the second value. Further, the predetermined column of the respective slice may be different from the central, for example middle, column.

The determination of the line-by-line deviation may include a normalization. For example, a suitable normalization may be based on the values and/or the number of voxels of the respective line on the first side or the second side of the predetermined column. For example, quadratic normalization may be applied for the determination of the line-by-line deviation. Normalization may be advantageous for the determination of the line-by-line deviation if the content of the respective slice has changed after an iteration step.

In addition, the determination of the line-by-line deviation may include masking and/or filtering of the respective slice. It is, for example, possible for edges and/or corners and/or high-contrast objects, for example bones, that are mapped in the respective slice, to be retained.

The correction of the first three-dimensional computed tomography dataset may include at least one rotation and/or translation of the first three-dimensional computed tomography dataset with respect to the respective predetermined and/or specified slice. The at least one rotation and/or translation may be determined using the at least one rotation parameter and/or the at least one translation parameter. Further, the correction of the first three-dimensional computed tomography dataset may include re-rasterization.

In a further embodiment of the method, the method for identifying stroke indications may be based on artificial intelligence. The method for identifying stroke indications may be trained by a machine learning method. The method for identifying stroke indications may include a trained function for identifying stroke indications. For example, the method for identifying stroke indications may be a neural network, for example a convolutional neural network (CNN) or a network including a convolutional layer.

A trained function maps input data to output data. The output data may, for example, furthermore be dependent upon one or more parameters of the trained function. The one or more parameters of the trained function may be determined and/or adapted by training. The determination and/or the adaptation of the one or more parameters of the trained function may, for example, be based on a pair made up of training input data and associated training output data. The trained function for creating training mapping data is applied to the training input data. For example, the determination and/or the adaptation may be based on a comparison of the training mapping data and the training output data. In general, a trainable function, i.e. a function with one or more parameters that have not yet been adapted, is also designated a trained function.

Other terms for trained function are trained mapping specification, mapping specification with trained parameters, function with trained parameters, method based on artificial intelligence, machine learning method. An example of a trained function is an artificial neural network. The edge weights of the artificial neural network correspond to the parameters of the trained function. Instead of the term “neural network”, it is also possible to use the term “neural net”. For example, a trained function may also be a deep artificial neural network (deep neural network). A further example for a trained function is a “support vector machine”, furthermore, for example, other machine learning methods may be used as the trained function.

The hemorrhagic and/or ischemic stroke indications may be determined by applying the method for identifying stroke indications, for example including a trained function for identifying stroke indications, to input data. The input data may be based on the artifact-reduced image dataset. Further, the training may, for example, take place by supervised training.

The trained function for identifying stroke indications may be trained by training datasets, wherein a training dataset in each case includes input data based on an artifact-reduced training image dataset. Further, the training datasets may in each case include comparison stroke indications corresponding to the respective artifact-reduced training image dataset. The comparison stroke indications, for example, correspond to all, for example ischemic and/or hemorrhagic, stroke indications contained in the artifact-reduced training image dataset. At least one parameter of the trained function for identifying stroke indications may be based on a comparison of stroke indications determined by applying the trained function for identifying stroke indications to the artifact-reduced training image dataset with the corresponding comparison stroke indications.

In a further embodiment of the method, the creation of the evaluation dataset may be based on artificial intelligence. A method for evaluating a manifestation of the identified stroke indications may be trained by a machine learning method. The method for evaluating a manifestation of the identified stroke indications may include a trained function for evaluating a manifestation of the identified stroke indications. For example, the method for evaluating a manifestation of the identified stroke indications may be a neural network, for example a convolutional neural network (CNN) or a network including a convolutional layer.

The evaluation dataset may be determined by applying the method for evaluating a manifestation of the identified stroke indications, for example including a trained function for evaluating a manifestation of the identified stroke indications, to input data. The input data may be based on the identified stroke indications. Further, the training may, for example, take place by supervised training. The trained function for evaluating a manifestation of the identified stroke indications may be trained by training datasets. A training dataset may in each case include input data based on training stroke indications. Further, the training datasets may in each case include a comparison evaluation dataset corresponding to the training stroke indications. The comparison evaluation dataset corresponds to an evaluation dataset that includes information, for example assigned values and/or value tuples, for evaluating the manifestation of all training stroke indications. At least one parameter of the trained function for evaluating a manifestation of the identified stroke indications may be based on a comparison of an evaluation dataset that is determined by applying the trained function for evaluating a manifestation of the identified stroke indications to the training stroke indications, with the corresponding comparison evaluation dataset.

The stroke indications may be identified by applying a provided method for identifying stroke indications to an artifact-reduced image dataset.

The output data of the method for identifying stroke indications to an artifact-reduced image dataset may in turn be used as input data for the method for evaluating a manifestation of the identified stroke indications.

In addition, the input data of the method for evaluating a manifestation of the identified stroke indications may additionally be based on the artifact-reduced image dataset. A training dataset may in each case include input data based on an artifact-reduced training image dataset. The training stroke indications may correspond to all the stroke indications contained in the artifact-reduced training image dataset.

In a further embodiment of the method, a thrombus may be identified as a stroke symptom. Input data of the method for evaluating the manifestation of the identified stroke indications may be based on the artifact-reduced image dataset and at least one tissue parameter of the thrombus. Further, the tissue parameter of the thrombus may be determined independently of the first three-dimensional computed tomography dataset.

The at least one tissue parameter of the thrombus may, for example, include a chemical composition and/or a density and/or a solubility. Further, the at least one tissue parameter of the thrombus, may for example in terms of time, be determined independently of the first three-dimensional computed tomography dataset, for example by a laboratory blood test and/or a spectroscopic examination and/or an imaging method, for example magnetic resonance imaging and/or computed tomography and/or a position emission tomography and/or a, for example a dual-source, X-ray examination of the examination object. Chemical and/or physical and/or physiological properties of the thrombus may also be ascertained indirectly using at least one tissue parameter of the examination object, for example a blood parameter.

The addition of the at least one tissue parameter of the thrombus, for example additionally to the artifact-reduced image dataset, as input data for the method for evaluating the manifestation of the identified stroke indications may provide increased accuracy of the evaluation of the manifestation of the identified stroke indications. Further, the at least one tissue parameter of the thrombus may provide further, for example chemical and/or physical and/or physiological, properties of the thrombus, that for example cannot be detected by a computed tomography examination, to be used as input data for the method for evaluating the manifestation of the identified stroke indications.

Further, in the case of a plurality of stroke indications identified as thrombi in the artifact-reduced image dataset, the input data of the method for evaluating the manifestation of the identified stroke indications may additionally be based on at least one tissue parameter of the respective thrombus in each case.

In a further embodiment of the method, output data of the method for evaluating the manifestation of the identified stroke indications may include a prognosis and/or a workflow note.

The training of the method for evaluating the manifestation of the identified stroke indications, for example including a trained function for evaluating a manifestation of the identified stroke indications, may take place by supervised training. The trained function for evaluating a manifestation of the identified stroke indications may be trained by training datasets. A training dataset may in each case include input data based on training stroke indications. If the training stroke indications include a thrombus, the input data may additionally be based on at least one tissue parameter of the thrombus.

Further, the training datasets may in each case include a comparison prognosis and/or a comparison workflow note. The comparison prognosis and/or the comparison workflow note in each case corresponds to at least one training stroke symptom.

The comparison prognosis corresponds to a temporal course of tissue parameters, for example of tissue adjacent to the training stroke indications. For example, the comparison prognosis may include a temporal course of a blood perfusion and/or a temporal course of a spatial extent of a necrosis and/or penumbra. For example, the comparison prognosis includes a temporal course of the tissue parameters, for example of tissue adjacent to the training stroke indications, as a physiological consequence of the respective training stroke symptom.

Further, the input data of the method for evaluating a manifestation of the identified stroke indications may be additionally based on physiological, real-time values from the examination object, for example during the recording of the plurality of two-dimensional X-ray projection images. The real-time values from the examination object may, for example, include a pulse value and/or a blood pressure and/or a temporal dimension for a period since the first occurrence of stroke indications and/or a value for a manifestation of the stroke, for example according to the stroke scale of the National Institute of Health (NIH Stroke Scale) and/or according to the modified Rankin-Skala (MRS).

In addition, the comparison workflow note may correspond to an instruction to an operator. The instruction to the operator may include a note relating to a suitable surgical instrument for removing and/or reducing the identified stroke symptom and/or a further imaging modality, for example a suitable contrast medium for improved depiction of the identified stroke symptom and/or a drug, for example for the resolution of the stroke symptom. Further, the instruction to the operator may include a temporal course of a plurality of notes.

If the output data of the method for evaluating a manifestation of the identified stroke indications includes the prognosis and the workflow note, the prognosis may include a temporal course of tissue parameters, for example of tissue adjacent to the training stroke indications, during and/or after the application of the workflow note.

In a further embodiment of the method, reconstruction may include at least one second three-dimensional computed tomography dataset. The at least one second computed tomography dataset may be reconstructed from a plurality of two-dimensional X-ray projection images recorded with different acquisition geometries from the examination object by a medical X-ray device. Further, provision of at least one further artifact-reduced image dataset may take place using the at least one second computed tomography dataset. The provision may include applying a further method for reducing artifacts. Further, a three-dimensional vessel image may be created. The creation of the three-dimensional vessel image may include segmentation of vessels from the at least one further artifact-reduced image dataset. In addition, vascular occlusions may be identified by applying a method for identifying vascular occlusions to the three-dimensional vessel image. Further, a plurality of maximum intensity projection images (MIP) may be created using the three-dimensional vessel image. Moreover, a further evaluation dataset may be created by applying a method for evaluating a manifestation of the identified vascular occlusions to the maximum intensity projection images. Moreover, the further evaluation dataset may be provided.

The medical X-ray device by which the plurality of two-dimensional X-ray projection images for reconstructing the at least one second three-dimensional computed tomography dataset were recorded may be different from the medical X-ray device for recording the plurality of two-dimensional X-ray projection images for reconstructing the first three-dimensional computed tomography dataset or correspond thereto.

Further, the medical X-ray device may be configured to record the plurality of two-dimensional X-ray projection images with different acquisition geometries from one another, for example about a common axis of rotation, from the examination object. For example, the common axis of rotation may correspond to a longitudinal axis of the examination object and/or a patient positioning facility on which the examination object is arranged.

A plurality of second three-dimensional computed tomography datasets may be reconstructed from the examination object. For example, the plurality of second three-dimensional computed tomography datasets may map a temporal course of a propagation movement of a contrast medium in the examination object.

The acquisition geometries may include an X-ray projection direction, for example relative to the examination object and/or a patient positioning facility, and/or an X-ray geometry, for example a cone beam geometry and/or fan beam geometry. As a result, redundancies, for example intersections and/or multiple scans of maps of regions of the examination object, may occur during the recording of the plurality of two-dimensional X-ray projection images. This may provide the reconstruction of the at least one second three-dimensional computed tomography dataset from the plurality of two-dimensional X-ray projection images, for example by an inverse Radon transform. The different acquisition geometries are configured such that a spatial region of a brain of the examination object is at least partially mapped by the plurality of two-dimensional X-ray projection images. In addition, the different acquisition geometries are configured to map a section of the examination object that is at least partially the same as the section of the examination object mapped in the first three-dimensional computed tomography dataset.

In addition, the method may include the recording of the plurality of two-dimensional X-ray projection images for reconstructing the at least one second three-dimensional computed tomography dataset by the medical X-ray device. Further, the method may include reception of the plurality of two-dimensional X-ray projection images for reconstructing the at least one second three-dimensional computed tomography dataset. The reception of the plurality of two-dimensional X-ray projection images for reconstructing the at least one second three-dimensional computed tomography dataset may include the acquisition and/or reading of a computer-readable storage medium and/or reception from a data storage unit, for example a database.

Further, the reconstruction of the at least one second three-dimensional computed tomography dataset may take place in dependence on the identified stroke indications and/or the evaluation dataset.

The application of the further method for reducing artifacts to the at least one second computed tomography dataset for example provides all disruptive image portions, that may mask or fog diagnostic image portions, to be removed. Artifacts may include disruptive image portions in the at least one second three-dimensional computed tomography dataset, for example noise and/or metal artifacts and/or motion artifacts.

Further, the diagnostic image portions are retained in the at least one further artifact-reduced image dataset provided. In addition, the at least one further artifact-reduced image dataset is three-dimensional and includes the same spatial resolution as the at least one second computed tomography dataset.

The provision of the at least one further artifact-reduced image dataset may for example include storage on a computer-readable storage medium and/or displaying on a depiction unit and/or transfer to a processing unit.

The creation of the three-dimensional vessel image may include segmentation of vessels from the at least one further artifact-reduced image dataset. The segmentation may for example take place based on values of voxels of the at least one further artifact-reduced image dataset. For example, at least one threshold value may be specified, wherein all voxels with a value below and/or above the at least one specified threshold value may be segmented from the further artifact-reduced image dataset as a vessel voxel. Further, a value interval may be specified. All voxels with a value within and/or outside the specified value interval may be segmented from the further artifact-reduced image dataset as a vessel voxel. A vessel voxel describes a voxel, that at least partially spatially maps a vessel and/or a section of a vessel.

In addition, the segmentation may include a method known from the prior art for vessel segmentation. It is for example possible for a shape and/or branching of the mappings of vessels contained in the further artifact-reduced image dataset to be used for the segmentation thereof.

The three-dimensional vessel image includes information on the spatial arrangement of vessels, that are mapped in the at least one further artifact-reduced image dataset. The information on the spatial arrangement of the vessels may for example include at least one centerline for each vessel and/or information on extension and/or information on branching and/or information on the course. For example, the three-dimensional vessel image may include a, for example abstract, three-dimensional vascular tree. Further, the three-dimensional vessel image may include information on perfusion for each of the vessels and/or vessel sections contained therein. The information on perfusion may, for example, be ascertained using a contrast, that is for example produced by a contrast medium in the examination object, in the further artifact-reduced image dataset. For example, information on perfusion may in each case be assigned to individual vessel sections contained in the three-dimensional vessel image.

Further, the three-dimensional vessel image may include a plurality of voxels. Each of the plurality of voxels may in each case be assigned a value, that for example corresponds to a contrast, for example with respect to the information on perfusion. In addition, each of the voxels of the three-dimensional vessel image may in each case be assigned a value corresponding to a property of a vessel that is at least partially mapped and/or contained in the voxel. For example, each of the voxels of the three-dimensional vessel image may in each case be assigned a color value, for example an RGB value and/or a gray value, in dependence on a property of the vessel that is at least partially mapped and/or contained therein. The property of the vessel may, for example, include information on the extent and/or information on branching and/or information on the course and/or information on perfusion.

In addition, the method may include a display of a depiction of the three-dimensional vessel image on a depiction unit, for example a display and/or monitor. The depiction of the three-dimensional vessel image may take place in dependence on the values assigned to the voxels of the three-dimensional vessel image.

The identification of the vascular occlusions by applying the method for identifying vascular occlusions may include a determination of a spatial extent and/or a spatial course and/or a position and/or an alignment and/or a morphological condition and/or a density of the respective vascular occlusion in the further artifact-reduced image dataset. The vascular occlusions may be identified using features of the three-dimensional vessel image, that are for example directly and/or implicitly characteristic of a vascular occlusion, for example a thrombus and/or a “large vessel occlusion” (LVO). For example, information on perfusion contained in the three-dimensional vessel image may be used to determine hypoperfused vessels and/or vessel sections.

Each of the plurality of maximum intensity projection images may for example include a projection of the three-dimensional vessel image along a predetermined projection direction in each case. The plurality of maximum intensity projection images are in each case two-dimensional. In the case of the creation of a maximum intensity projection image, it is possible in each case to determine a voxel of the three-dimensional vessel image along the predetermined projection direction corresponding to a voxel of the two-dimensional maximum intensity projection image, wherein the voxel determined includes maximum contrast, for example with respect to the information on perfusion. In addition, a three-dimensional maximum intensity projection dataset may be formed from the plurality of maximum intensity projection images.

Further, the method, that is applied to the plurality of maximum intensity projection images to create the further evaluation dataset may be configured to evaluate a manifestation of the identified vascular occlusions. The evaluation of the manifestation of the identified vascular occlusions may, for example, include an evaluation according to the “multiphase CT angiography collateral score in acute stroke” (mCTA collateral). For example, the evaluation of the manifestation of the identified vascular occlusions may include a comparison between two hemispheres of the brain of the examination object mapped in the plurality of maximum intensity projection images. The evaluation of the identified vascular occlusions may furthermore include an evaluation of the spatial extent and/or the spatial course and/or the position and/or the alignment and/or the morphological condition and/or the density of the respective vascular occlusion in the plurality of maximum intensity projection images. The evaluation of the manifestation of the identified vascular occlusions may include an assignment of a value, for example a dimension, and/or a value tuple to at least one, for example all, identified vascular occlusions.

For example, the further evaluation dataset may include a plurality of two-dimensional partial evaluation datasets, wherein each of the plurality of two-dimensional partial evaluation datasets includes an evaluation of the manifestation of the vascular occlusions identified in one of the plurality of maximum intensity projection images in each case. In addition, the further evaluation dataset may be created by applying the method for evaluating a manifestation of the identified vascular occlusions to the three-dimensional maximum intensity projection dataset. Further, the further evaluation dataset is three-dimensional.

The provision of the further evaluation dataset may for example include storage on a computer-readable storage medium and/or displaying on a depiction unit and/or transfer to a processing unit. The further evaluation dataset provided may for example be useful for diagnostic assistance for an operator, for example a physician.

In a further embodiment of the method, the further method for reducing artifacts may include the following steps: creation of at least one further three-dimensional auxiliary dataset by undersampling the at least one second three-dimensional computed tomography dataset; determination in each case of a slice along two spatial directions of the at least one further three-dimensional auxiliary dataset, wherein each of the two slices in each case includes a predetermined column and, in each case, at least one line; determination of at least one rotation parameter and/or at least one translation parameter in order for reducing a line-by-line deviation with respect to the in each case predetermined column in each of the two slices; and correction of the at least one second three-dimensional computed tomography dataset by applying the at least one rotation parameter and/or the at least one translation parameter.

The advantages of the further method for reducing artifacts substantially correspond to the advantages of the above-described embodiments of the method for reducing artifacts. Features, advantages, or alternative embodiments mentioned may also be transferred to the further method for reducing artifacts and vice versa.

In a further embodiment, the method may furthermore include a recording of at least one further two-dimensional X-ray projection image from the examination object by a medical X-ray device. Further, it is possible to determine a common slice in the first and the at least one second computed tomography dataset. The common slice may extend perpendicularly to the projection direction of the at least one further two-dimensional X-ray projection image. Further, a two-dimensional vessel slice image may be created from the three-dimensional vessel image along the common slice. In addition, a two-dimensional thrombus slice image may be created from the three-dimensional thrombus image along the common slice. Moreover, a two-dimensional superimposed image may be created. The creation of the two-dimensional superimposed image may include the superimposition of the vessel slice image and/or the thrombus slice image and the at least one further X-ray projection image. Further, the two-dimensional superimposed image may be provided.

The medical X-ray device by which the at least one further two-dimensional X-ray projection image is recorded may for example be different from the medical X-ray device for recording the plurality of two-dimensional X-ray projection images for reconstructing the first and/or the at least one second three-dimensional computed tomography dataset or correspond thereto.

Further, the at least one further two-dimensional X-ray projection image maps a section of the examination object, that is at least partially contained in the section of the examination object contained in the at least one second three-dimensional computed tomography dataset.

The determination of the common, for example two-dimensional, slice in the first and the at least one second computed tomography dataset may take place using operating parameters, for example information on the position and/or information on the alignment, and/or recording parameters of the medical X-ray device used in each case for recording and/or using image features, for example edges, and/or using anatomical features. The determination of the common slice is for example provided by the fact that the sections of the examination object mapped in the first and the at least one second three-dimensional computed tomography dataset are at least partially the same.

The fact that the common slice extends perpendicularly to the projection direction of the at least one further two-dimensional X-ray projection image provides it to be ensured that the at least one further two-dimensional X-ray projection image at least partially includes the, for example anatomical, features of the first and the at least one second three-dimensional computed tomography dataset, that are contained in the common slice.

The creation of the two-dimensional vessel slice image from the three-dimensional vessel image may include the selection and/or assignment of the voxels of the three-dimensional vessel image contained in the common slice. Further, the creation of the two-dimensional thrombus slice image from the three-dimensional thrombus image may include the selection and/or assignment of the voxels of the three-dimensional thrombus image contained in the common slice.

Further, the superimposition of the vessel slice image and/or the thrombus slice image and of the at least one further X-ray projection image may include, for example weighted averaging. Moreover, the superimposition may take place in sections and/or in regions along the common slice. Further, the superimposition may take place in dependence on a specified threshold value of a value of voxels of the vessel slice image and/or the thrombus slice image and/or the at least one further X-ray projection image.

The provision of the two-dimensional superimposed image may for example include storage on a computer-readable storage medium and/or displaying on a depiction unit and/or transfer to a processing unit.

In a further embodiment of the method, the determination of the common slice in the first and the at least one second computed tomography dataset may include registration. The first and the at least one second computed tomography dataset may be registered to one another and/or, for example in each case, to the at least one further two-dimensional X-ray projection image. The registration may for example take place with respect to the common slice. If the first and the at least one second computed tomography dataset are registered to one another, the registration may take place three-dimensionally.

Further, the registration may include rigid and/or non-rigid transformation and/or interpolation and/or re-rasterization. Moreover, the registration may be based on operating parameters and/or recording parameters of the medical X-ray device used in each case for recording and/or using image features, for example edges, and/or using anatomical features.

The registration may provide the reduction of motion artifacts, that may for example be caused by motion of the examination object during and/or between the recording of the plurality of two-dimensional X-ray projection images and/or the at least one further two-dimensional X-ray projection image.

In a further embodiment of the method, the further method for reducing artifacts may include registering the two-dimensional X-ray projection images to one another and/or applying a filter for noise reduction and/or motion correction.

The advantages of the further method for reducing artifacts substantially correspond to the advantages of the above-described embodiments of the method for reducing artifacts. Features, advantages, or alternative embodiments mentioned herein may also be transferred to the further method for reducing artifacts and vice versa.

In a further embodiment of the method, a plurality of, for example three, second three-dimensional computed tomography datasets may be recorded of the examination object in temporal succession. A phase of a perfusion may be assigned to each of the second computed tomography datasets. For example, the plurality of second three-dimensional computed tomography datasets may be reconstructed from a plurality of two-dimensional X-ray projection images by at least two rotation runs of the medical X-ray device. A rotation speed and/or recording frequency and recording of the two-dimensional X-ray projection images may be specified such that a physiological process, for example a temporal course of a blood perfusion and/or a contrast medium perfusion, in the examination object may be acquired.

Further, the assignment of the phases of the perfusion, for example a blood perfusion and/or a contrast medium perfusion, may take place in specified temporal intervals. The temporal intervals may for example in each case be specified as different from one another. This may enable a particularly good acquisition of respective phase of the perfusion by the respective second three-dimensional computed tomography dataset. Further, a subdivision of the phases of the perfusion may take place based on an external trigger signal, for example an electrocardiogram and/or an electromyogram and/or a breathing sensor and/or a motion sensor. The external trigger signal may map at least one physiological parameter of the examination object during the recording of the plurality of two-dimensional X-ray projection images.

In addition, the temporal intervals may in each case be specified as the same. This may enable acquisition of the perfusion in the examination object that is particularly uniform in terms of time.

In a further embodiment of the method, the creation of the three-dimensional vessel image may include an assignment of an image value to each of the vessels in the three-dimensional vessel image, wherein the image values may be determined by the phases of the plurality of second computed tomography datasets. This provides it to be achieved that the image values of the three-dimensional vessel image may include the temporal course of the phases of the perfusion. For example, the determination of the image values using the phases of the plurality of second computed tomography datasets may include the assignment of a tuple to each of the vessels in the vessel image, for example a color value, for example an RGB value and/or a gray value. Further, the image values may be determined in dependence on a threshold value of the respective phase of the perfusion being undershot or exceeded in each case. This, for example, provides vessels that only have high contrast or low contrast in individual phases to be highlighted in the three-dimensional vessel image. For example, perfused and/or re-perfused vessels may be highlighted in the three-dimensional vessel image. Further, the determination of the image values by the phases of the plurality of second computed tomography datasets provides hypoperfused vessels in the vessel image to be demarcated particularly well from, for example normal, perfused vessels.

According to an above-described embodiment of the method, the values of the voxels of the three-dimensional vessel image may be determined in dependence on the image values assigned to the vessels that are least partially contained in the voxels. Herein, partial volume effects may be taken into account.

In a further embodiment of the method, the further method for reducing artifacts may include creation of at least one three-dimensional difference image data set. To create the difference image data set, a three-dimensional mask image may be subtracted from at least one of the plurality of second three-dimensional computed tomography datasets.

This, for example, provides a tissue background and/or a noise and/or at least one non-moving region to be removed from the plurality of second three-dimensional computed tomography datasets. A moving region, for example a moving vessel section, of the examination object may for example be identified from a temporal course of a movement of a surgical instrument on and/or in the examination object and/or a temporal course of a blood perfusion and/or a temporal course of a contrast medium perfusion. Further, a non-moving region, for example a vessel section and/or tissue section, of the examination object may be formed by an unchanged region of the examination object in the temporal course.

The three-dimensional mask image may, for example, be created from the first three-dimensional computed tomography dataset and/or from the artifact-reduced image dataset. Further, the three-dimensional mask image may be created from one of the plurality of second three-dimensional computed tomography datasets. In addition, the three-dimensional mask image may for example be created synthetically using a recording of the examination object that is independent of the recording of the plurality of two-dimensional X-ray projection images for reconstructing the first and the plurality of second computed tomography datasets. The three-dimensional mask image may be recorded by a further imaging modality, for example magnetic resonance tomography and/or positron emission tomography and/or an ultrasound examination. Further, the three-dimensional mask image may be created by, for example weighted, averaging of the plurality of three-dimensional computed tomography datasets.

The subtraction of the three-dimensional mask image from the at least one of the plurality of second three-dimensional computed tomography datasets may take place restricted to individual voxels and/or regions of the at least one of the plurality of second three-dimensional computed tomography datasets. For example, the subtraction may only be applied to voxels of the at least one of the plurality of second three-dimensional computed tomography datasets with a value below and/or above a specified threshold value. This provides predetermined anatomical structures to be retained in the three-dimensional difference image data set. The subtraction of the mask image is not applied to voxels of the at least one of the plurality of second three-dimensional computed tomography datasets that map the predetermined anatomical structures.

In a further embodiment of the method, the identification of the vascular occlusions in the three-dimensional vessel image may be based on artificial intelligence. The method for identifying vascular occlusions may be trained by a machine learning method. The method for identifying vascular occlusions may include a trained function for identifying vascular occlusions. For example, the method for identifying vascular occlusions may be a neural network, for example a convolutional neural network (CNN) or a network including a convolutional layer.

The vascular occlusions may be determined by applying the method for identifying vascular occlusions, for example including a trained function for identifying vascular occlusions, to input data. The input data may be based on the three-dimensional vessel image. Further, the training may for example take place by supervised training.

The trained function for identifying vascular occlusions may be trained by training datasets. A training dataset in each case includes input data based on a three-dimensional training vessel image. Further, the training datasets may in each case include a comparison vessel occlusion dataset corresponding to the respective three-dimensional training vessel image. The comparison vessel occlusion dataset may include information, for example information on the spatial extent and/or information on the position and/or information on the alignment, of all the vascular occlusions contained in the three-dimensional training vessel image. The vascular occlusions contained in the comparison vessel occlusion dataset for example correspond to all the vascular occlusions contained in the three-dimensional training vessel image. At least one parameter of the trained function for identifying vascular occlusions may be based on a comparison of identified vascular occlusions that are determined by applying the trained function for identifying vascular occlusions to the three-dimensional training vessel image with the vascular occlusions contained in the corresponding comparison vessel occlusion dataset.

In a further embodiment of the method, the creation of the further evaluation dataset may be based on artificial intelligence. The method for evaluating a manifestation of the identified vascular occlusions may be trained by a machine learning method. The method for evaluating a manifestation of the identified vascular occlusions may include a trained function for evaluating a manifestation of the identified vascular occlusions. For example, the method for evaluating a manifestation of the identified vascular occlusions may be a neural network, for example a convolutional neural network (CNN) or a network including a convolutional layer.

The further evaluation dataset may be determined by applying the method for evaluating a manifestation of the identified vascular occlusions, for example including a trained function for evaluating a manifestation of the identified vascular occlusions, to input data. The input data may be based on the maximum intensity projection images. Further, the training may for example take place by supervised training.

The trained function for evaluating a manifestation of the identified vascular occlusions may be trained by training datasets, wherein a training dataset may in each case include input data based on training maximum intensity projection images. Further, the training datasets may in each case include a further comparison evaluation dataset corresponding to the training maximum intensity projection images. The further comparison evaluation dataset corresponds to a further evaluation dataset, that includes information, for example assigned values and/or value tuples, for evaluating the manifestation of all the vascular occlusions mapped in the maximum intensity projection images. At least one parameter of the trained function for evaluating a manifestation of the identified vascular occlusions may be based on a comparison of a further evaluation dataset, that is determined by applying the trained function for evaluating a manifestation of the identified vascular occlusions to the training maximum intensity projection images, with the corresponding further comparison evaluation dataset.

The vascular occlusions may be identified by applying a method for identifying vascular occlusions to a three-dimensional vessel image.

In addition, the input data method for evaluating a manifestation of the identified vascular occlusions may additionally be based on the identified vascular occlusions. A training dataset may in each case include input data based on a training vessel occlusion dataset. The at least one vascular occlusion may be identified by applying the method for identifying vascular occlusions to a three-dimensional training vessel image. Further, the plurality of training maximum intensity projection images may be created using the three-dimensional training vessel image. Further, the further comparison evaluation dataset may include information for evaluating the manifestation of all the vascular occlusions contained in the training vessel occlusion dataset.

Further, a medical X-ray device is provided that is configured to carry out a method for providing an evaluation dataset from a first medical three-dimensional computed tomography dataset. Such a, for example medical, X-ray device may be configured as a C-arm X-ray device and/or as a computed tomography system (CT). Further, the X-ray device may be configured to record a plurality of two-dimensional X-ray projection images with different acquisition geometries from an examination object. Hereinafter, the first three-dimensional computed tomography dataset may be reconstructed from the plurality of two-dimensional X-ray projection images with different acquisition geometries. In addition, an artifact-reduced image dataset may be provided. The provision may include applying a method for reducing artifacts to the first computed tomography dataset. Further, it is possible to identify for example hemorrhagic and/or ischemic stroke indications by applying a method for identifying stroke indications to the artifact-reduced image dataset. Moreover, an evaluation dataset may be created by applying a method for evaluating a manifestation of the identified stroke indications to the artifact-reduced image dataset. In addition, the evaluation dataset may be provided.

Further, a processing unit, for example a microprocessor is provided that is configured to process information and/or data and/or signals from the X-ray device and/or further components. Further, the processing unit is configured to send control commands to the X-ray device and/or its constituent parts and/or further components. In addition, the medical X-ray device, for example the processing unit, may be configured to receive the plurality of two-dimensional X-ray projection images for reconstructing the first three-dimensional computed tomography dataset. Further, the medical X-ray device, for example the processing unit, may be configured to receive the plurality of two-dimensional computed tomography datasets for reconstructing the at least one second three-dimensional computed tomography dataset. The reception of the two-dimensional X-ray projection images may take place by an interface, for example by a computer-readable storage medium and/or a database.

The X-ray device may include a depiction unit, for example a display and/or a monitor, that is configured to display information and/or graphical depictions of information from the X-ray device and/or further components. For example, the depiction unit may be configured to display a graphical depiction of the evaluation dataset and/or the further evaluation dataset.

The advantages of the X-ray device substantially correspond to the advantages of the method for providing an evaluation dataset from a first medical three-dimensional computed tomography dataset. Features, advantages, or alternative embodiments mentioned herein may also be transferred to the other claimed subject matter and vice versa.

Further, a computer program product is provided, that includes a program and may be loaded directly into a memory of a programmable computing unit, and programming, for example libraries and auxiliary functions to carry out a method for providing an evaluation dataset from a first medical three-dimensional computed tomography dataset when the computer program product is executed. The computer program product may include software with a source code that still has to be compiled and linked or only has to be interpreted or executable software code that only needs to be loaded into the processing unit for execution. The computer program product provides the method for providing an evaluation dataset from a first medical three-dimensional computed tomography dataset to be carried out quickly, identically repeatably and robustly. The computer program product is configured such that it may carry out the method steps by the processing unit. The processing unit must in each case fulfil the requisite conditions such as, for example, including an appropriate random-access memory, an appropriate graphics card or a corresponding logic unit so that the respective method steps may be carried out efficiently.

The computer program product is for example stored on a computer-readable storage medium or held resident on a network or server from where it may be loaded into the processor of a processing unit, that is directly connected to the processing unit or may be configured as part of the processing unit. Furthermore, control information of the computer program product may be stored on an electronically readable data carrier. The control information of the electronically readable data carrier may be configured to carry out a method when the data carrier is used in a processing unit. Examples of electronically readable data carriers are a DVD, a magnetic tape, or a USB stick on which electronically readable control information, for example software, is stored. When the control information is read from the data carrier and stored in a processing unit, all the embodiments of the above-described methods may be carried out. Embodiments may also be based on the computer-readable storage medium and/or the electronically readable data carrier.

### BRIEF DESCRIPTION OF THE FIGURES

FIG. 1 depicts an example embodiment of a method for providing an evaluation dataset from a first medical three-dimensional computed tomography dataset.

FIG. 2 depicts an example embodiment of a method for providing an evaluation dataset from a first medical three-dimensional computed tomography dataset.

FIG. 3 depicts an example embodiment of a method for providing an evaluation dataset from a first medical three-dimensional computed tomography dataset.

FIG. 4 depicts an example embodiment of a method for providing an evaluation dataset from a first medical three-dimensional computed tomography dataset.

FIG. 5 depicts an example embodiment of a method for providing an evaluation dataset from a first medical three-dimensional computed tomography dataset.

FIG. 6 depicts an example embodiment of a method for providing an evaluation dataset from a first medical three-dimensional computed tomography dataset.

FIG. 7 depicts an example embodiment of a method for providing an evaluation dataset from a first medical three-dimensional computed tomography dataset.

FIG. 8 depicts an embodiment of a method for reducing artifacts.

FIG. 9 depicts an embodiment of a medical C-arm X-ray device for performing a method.

### DETAILED DESCRIPTION

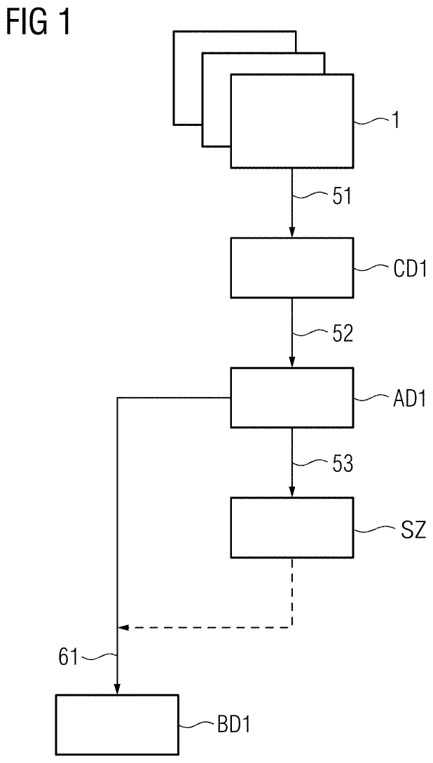

FIG. 1 is a schematic depiction of an embodiment of the method for providing an evaluation dataset from a first medical three-dimensional computed tomography dataset. The first three-dimensional computed tomography dataset CD1 may be reconstructed 51 from a plurality of two-dimensional X-ray projection images 1 recorded with different acquisition geometries from an examination object by a medical X-ray device. Further, an artifact-reduced image dataset AD1 may be provided. The provision may include applying a method for reducing artifacts 52 to the first computed tomography dataset CD1. In addition, for example hemorrhagic and/or ischemic stroke indications SZ may be identified by applying a method for identifying stroke indications 53 to the artifact-reduced image dataset AD1. Further, an evaluation dataset BD1 may be created 61 by applying a method for evaluating a manifestation of the identified stroke indications SZ to the artifact-reduced image dataset AD1. After this, the evaluation dataset BD1 may be provided.

The method for reducing artifacts may include registering the plurality of two-dimensional X-ray projection images 1 to one another and/or applying a filter for noise reduction and/or motion correction.

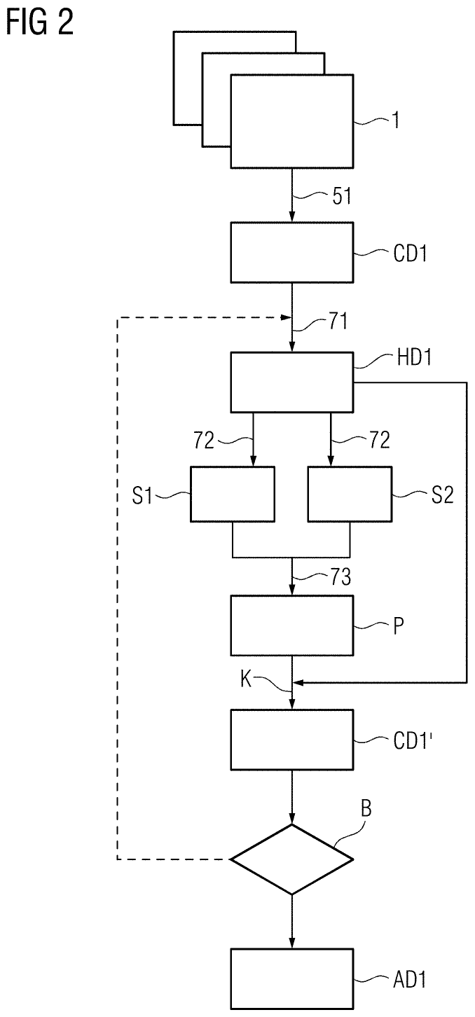

FIG. 2 depicts a further embodiment of the method. The method for reducing artifacts may include creation 71 of a three-dimensional auxiliary dataset HD1 by undersampling the first three-dimensional computed tomography dataset CD1. Further, in each case a slice S1 and S2 may be determined 72 along two spatial directions of the three-dimensional auxiliary dataset HD1. Each of the two slices S1 and S2 may in each case include a predetermined column and, in each case, at least one line. Further, at least one rotation parameter P and/or at least one translation parameter P for reducing a line-by-line deviation with respect to the predetermined column in each case may be determined 73 in each of the two slices S1 and S2. After this, the first three-dimensional computed tomography dataset CD1 may be corrected K by applying the at least one rotation parameter P and/or the at least one translation parameter P.

The determination of the at least one rotation parameter P and/or the at least one translation parameter P may take place iteratively. The determination of the at least one rotation parameter P and/or the at least one translation parameter P may include an iterative correction K of the three-dimensional auxiliary dataset HD1 by applying the most recently determined 73 at least one rotation parameter P and/or at least one translation parameter P in each case. The correction K may include at least one rotation and/or translation of the three-dimensional auxiliary dataset HD1 with respect to the respective predetermined and/or specified slice S1 or S2. Further, the correction K of the three-dimensional auxiliary dataset HD1 may include re-rasterization for example to the same spatial resolution as the first three-dimensional computer dataset CD1.

After each iteration step, the correction K may create a three-dimensional intermediate dataset CD1′. An iteration criterion B, that may, for example, include a maximum number of iteration steps and/or a quality criterion with respect to the line-by-line deviation within the in each case predetermined and/or specified slice S1 or S2, provides the determination of either a further iteration step and/or provision of the artifact-reduced image dataset AD1. The most recently generated three-dimensional intermediate dataset CD1 may be provided as an artifact-reduced image dataset AD1.

In addition, the method for identifying stroke indications may be based on artificial intelligence.

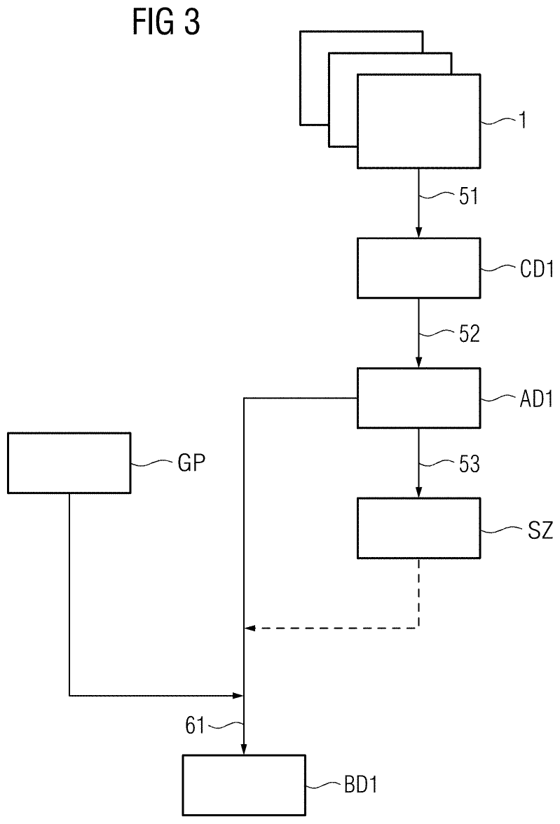

FIG. 3 is a schematic depiction of a further embodiment of the method. The creation 61 of the evaluation dataset BD1 may be based on artificial intelligence. A thrombus may be identified 53 as a stroke symptom SZ. Input data of the method for evaluating the manifestation of the identified stroke indications SZ is based on the artifact-reduced image dataset BD1 and at least one tissue parameter GP of the thrombus. Further, the tissue parameter GP of the thrombus may be determined independently of the first three-dimensional computed tomography dataset CD1.

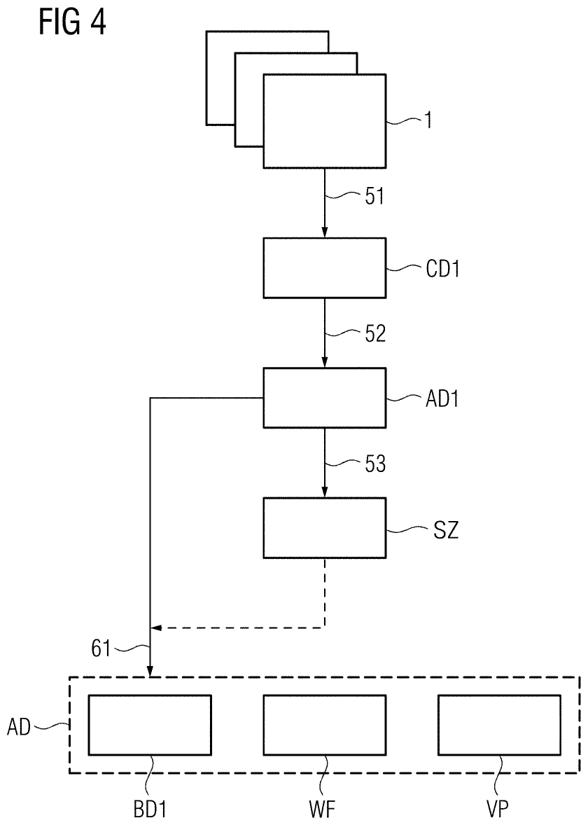

FIG. 4 is a schematic depiction of a further embodiment of the method. Output data AD of the method for evaluating the manifestation of the identified stroke indications SZ includes a prognosis VP and/or a workflow note WF.

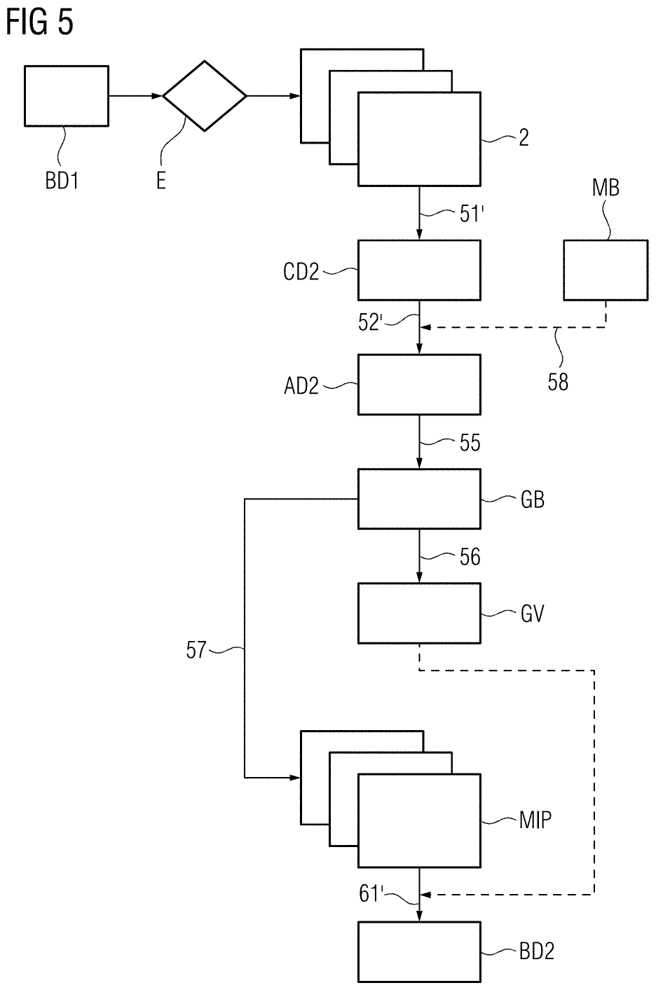

FIG. 5 is a schematic depiction of a further embodiment of the method. The method may after the creation of the evaluation dataset BD1 include reconstruction of at least one second three-dimensional computed tomography dataset CD2. The at least one second computed tomography dataset CD2 may be reconstructed from a plurality of two-dimensional X-ray projection images 2 recorded with different acquisition geometries from the examination object by a medical X-ray device. Further, at least one further artifact-reduced image dataset AD2 may be provided 51′ using the at least one second computed tomography dataset CD2. The provision may include applying a further method for reducing artifacts 52′. Moreover, a three-dimensional vessel image GB may be created 55. The creation 55 may include segmentation of vessels from the at least one further artifact-reduced image dataset AD2. Vascular occlusions GV may be identified by applying 56 a method for identifying vascular occlusions to the three-dimensional vessel image. In addition, a plurality of maximum intensity projection images MIP may be generated 57 using the three-dimensional vessel image GB. Further, a further evaluation dataset BD2 may be generated 61′ by applying a method for evaluating a manifestation of the identified vascular occlusions GV to the maximum intensity projection images MIP. After this, the further evaluation dataset BD2 may be provided.

In addition, the further method for reducing artifacts may include creation of at least one three-dimensional difference image data set (not shown). To create the three-dimensional difference image data set, a three-dimensional mask image MB may be subtracted 58 from at least one second three-dimensional computed tomography dataset CD2.

Moreover, the further method for reducing artifacts may include registering the two-dimensional X-ray projection images 2 for reconstructing the at least one second computed tomography dataset CD2 to one another and/or applying a filter for noise reduction and/or motion correction.

Further, the reconstruction of the at least one second three-dimensional computed tomography dataset CD2 may take place in dependence on the identified stroke indications SZ and/or the evaluation dataset BD1. The reconstruction of the at least one second three-dimensional computed tomography dataset CD2 may for example take place using a decision criterion E. The decision criterion E may, for example, be based on the ischemic and/or hemorrhagic stroke indications SZ identified on the basis of the first three-dimensional computed tomography dataset CD1 and/or an evaluation of the identified stroke indications SZ in the evaluation dataset BD1.

Further, a plurality of second three-dimensional computed tomography datasets CD2 may be recorded in temporal succession. A phase of a perfusion may be assigned to each of the second computed tomography datasets CD2. Moreover, the creation of the three-dimensional vessel image GB may include the assignment of an image value to each of the vessels in the three-dimensional vessel image GB. The image values may be determined by the phases of the plurality of second computed tomography datasets CD2.

In addition, the identification 56 of the vascular occlusions GV in the three-dimensional vessel image GB may be based on artificial intelligence.

Further, the creation 61′ of the further evaluation dataset BD2 may be based on artificial intelligence.

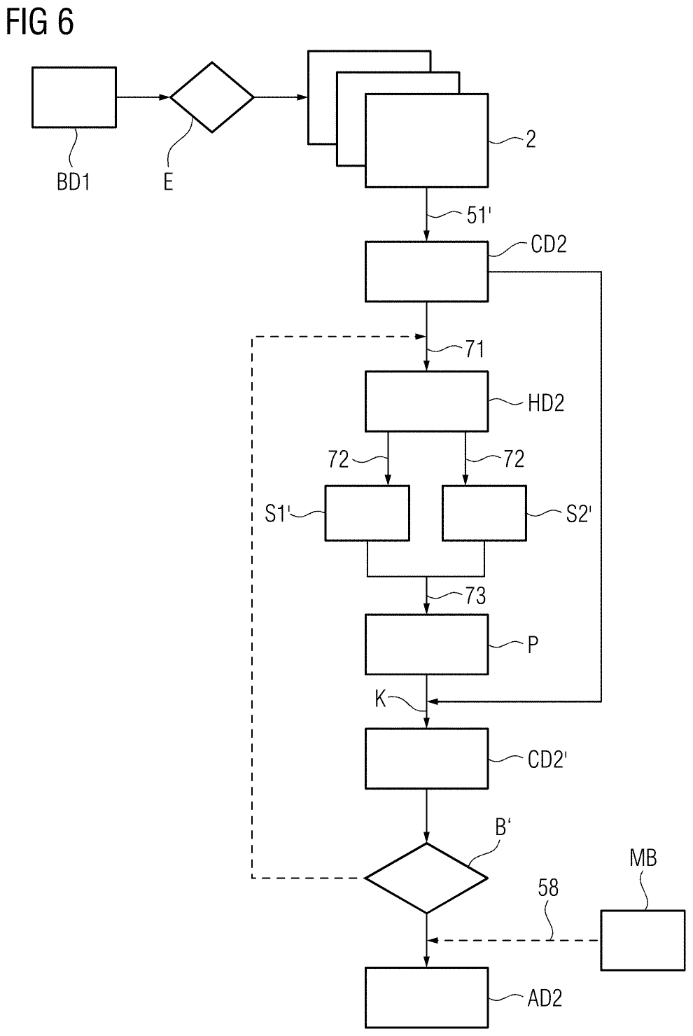

FIG. 6 is a schematic depiction of a further embodiment of the method. The further method for reducing artifacts may include creating 71 at least one further three-dimensional auxiliary dataset HD2 by undersampling the at least one second three-dimensional computed tomography dataset CD2. Further, in each case a slice S1′ and S2′ may be determined 72 along two spatial directions of the at least one further three-dimensional auxiliary dataset HD2. Each of the two slices S1′ and S2′ may in each case include a predetermined column and, in each case, at least one line. In addition, at least one rotation parameter P and/or at least one translation parameter P for reducing a line-by-line deviation with respect to the in each case predetermined column may be determined 73 in each of the two slices S1′ and S2′. Further, a correction K of the at least one second three-dimensional computed tomography dataset CD2 may take place by applying the at least one rotation parameter P and/or the at least one translation parameter P.

The correction K may include at least one rotation and/or translation of the at least one further three-dimensional auxiliary dataset HD2 with respect to the respective predetermined and/or specified slice S1′ or S2′. Further, the correction K of the at least one further three-dimensional auxiliary dataset HD2 may include re-rasterization for example to the same spatial resolution as the at least one second three-dimensional computer dataset CD2.

After each iteration step, the correction K may create at least one further three-dimensional intermediate dataset CD2′. An iteration criterion B that may, for example, include a maximum number of iteration steps and/or quality criterion with respect to the line-by-line deviation within the in each case predetermined and/or specified slice S1′ or S2′, may provide the determination of either a further iteration step and/or a provision of the at least one further artifact-reduced image dataset AD2. The most recently generated at least one further three-dimensional intermediate dataset CDT may be provided as at least one further artifact-reduced image dataset AD2.

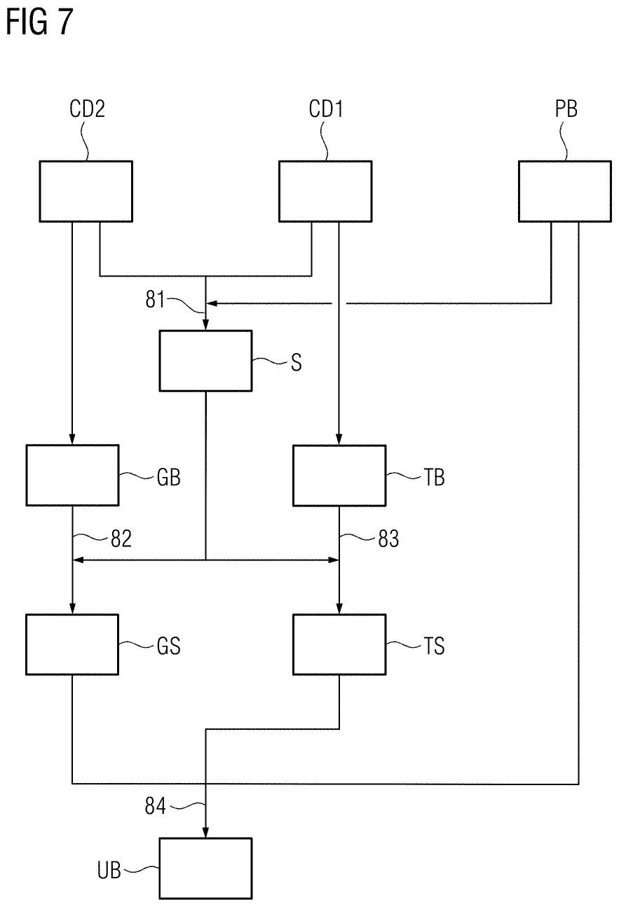

FIG. 7 is a schematic depiction of a further embodiment of the method. At least one further two-dimensional X-ray projection image PB may be recorded from the examination object by a medical X-ray device. Further, a common slice S may be determined 81 in the first computed tomography dataset CD1 and the at least one second computed tomography dataset CD2. The common slice may extend perpendicularly to the projection direction of the at least one further two-dimensional X-ray projection image PB. In addition, a two-dimensional vessel slice image GS may be created 82 from the three-dimensional vessel image GB along the common slice S. Not all the intermediate steps for creating the vessel image on the basis of the at least one second three-dimensional computed tomography dataset CD2 are depicted.

In addition, the method may include creating a three-dimensional TB from the artifact-reduced image dataset AD1 and/or the evaluation dataset BD1. A thrombus may be identified as a stroke symptom SZ. Further, the creation of the three-dimensional thrombus image TB may include segmentation of the thrombus from the artifact-reduced image dataset AD1 and/or the evaluation dataset BD1. After this, the three-dimensional thrombus image TB may be provided. Not all the intermediate steps for creating the thrombus image on the basis of the first three-dimensional computed tomography dataset CD1 are depicted. A two-dimensional thrombus slice image TS may be created 83 from the three-dimensional thrombus image TB along the common slice S.

Further, a two-dimensional superimposed image UB may be created 84. The creation 84 may include superimposition of the vessel slice image GS and/or the thrombus slice image TS and the at least one further X-ray projection image PB. The two-dimensional superimposed image UB may be provided.

In addition, the determination 81 of the common slice S in the first computed tomography dataset CD1 and the at least one second computed tomography dataset CD2 may include registration. The first computed tomography dataset CD1 and the at least one second computed tomography dataset CD2 may be registered to one another and/or, for example in each case, to the at least one further two-dimensional X-ray projection image PB.

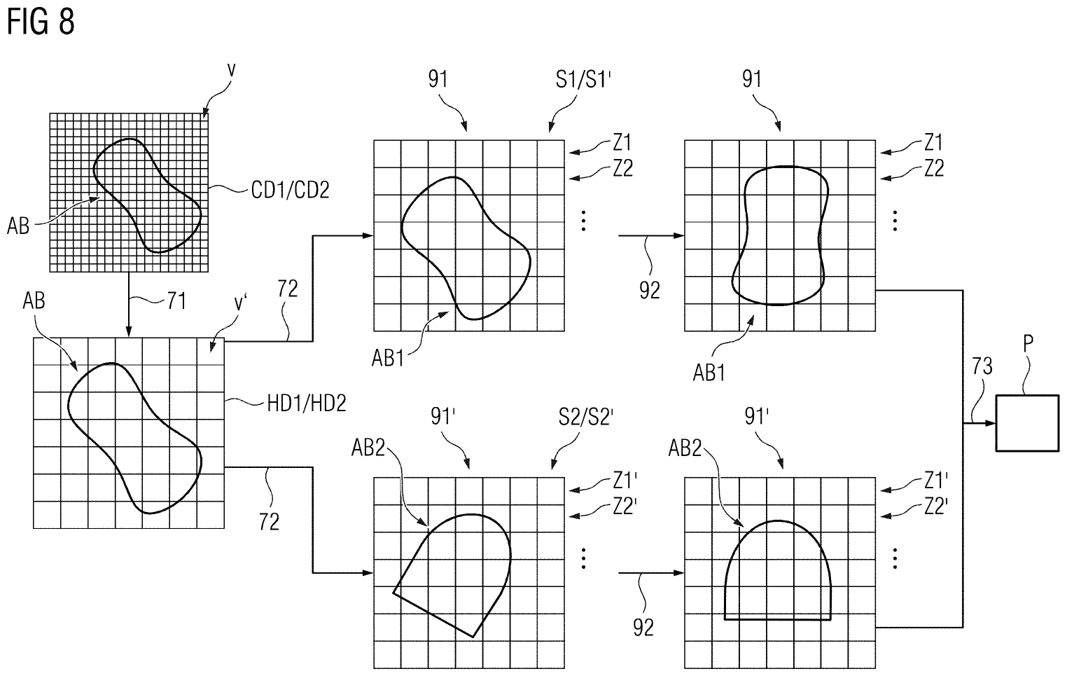

FIG. 8 is a schematic depiction of an embodiment of a method for reducing artifacts. The embodiment depicted may be applied for both the method for reducing artifacts and analogously for the further method for reducing artifacts.

The following describes a depicted embodiment for the method for reducing artifacts. In a first step, the three-dimensional auxiliary dataset HD1 may be created by undersampling 71 the first three-dimensional computed tomography dataset CD1. The first three-dimensional computed tomography dataset CD1 contains a map AB of the examination object. For example, the three-dimensional auxiliary dataset 71 may include a lower number of voxels v′ than the first three-dimensional computed tomography dataset CD1. Further, a spatial volume of the voxels v′ of the three-dimensional auxiliary dataset HD1 may be greater than a volume of the voxels v of the first three-dimensional computed tomography dataset CD1. Further, in each case a slice S1 and S2 may be determined 72 along two spatial directions of the three-dimensional auxiliary dataset HD1. Each of the two slices S1 and S2 may include in each case a predetermined column 91 and 91′ and in each case at least one line Z1, Z2 and Z1′, Z2′. Moreover, each of the two slices S1 and S2 may in each case include a map AB1 and AB2 of the examination object.

The determination of the at least one rotation parameter P and/or the at least one translation parameter P may include an iterative correction 92 of the three-dimensional auxiliary dataset HD1 by applying the in each case most recently determined at least one rotation parameter P and/or at least one translation parameter P. The line-by-line deviation with respect to the in each case predetermined column 91 and 91′ in each of the two slices S1 and S2 may be, for example iteratively, reduced. The most recently determined 73 at least one rotation parameter P and/or at least one translation parameter P may be used to correct K the first three-dimensional computed tomography dataset CD1.

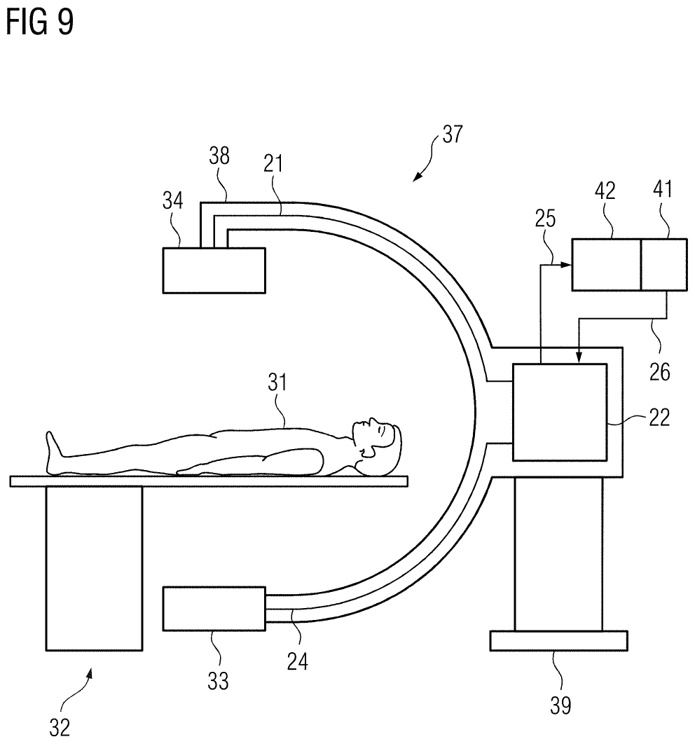

FIG. 9 is schematic depiction of a medical C-arm X-ray device 37, that is configured to carry out an embodiment of the method. The medical C-arm X-ray device 37 includes a detector unit 34, an X-ray source 33 and a processing unit 22. For recording the plurality of two-dimensional X-ray projection images 1 for reconstructing the first computed tomography dataset CD1 and/or the plurality of two-dimensional X-ray projection images 2 for reconstructing the at least one second computed tomography dataset CD2 and/or for recording the at least one further two-dimensional X-ray projection image PB, the arm 38 of the C-arm X-ray device may be mounted movably about one or more axes. This provides the plurality of two-dimensional X-ray projection images 1 for reconstructing the first computed tomography dataset CD1 and/or the plurality of two-dimensional X-ray projection images 2 for reconstructing the at least one second computed tomography dataset CD2 and/or the at least one further two-dimensional X-ray projection image PB to be recorded with in each case different, for example from one another, acquisition geometries. Further, the medical C-arm X-ray device 37 may include a moving mechanism 39 that provides movement of the C-arm X-ray device 37 in space.

For recording the plurality of two-dimensional X-ray projection images 1 for reconstructing the first computed tomography dataset CD1 and/or the plurality of two-dimensional X-ray projection images 2 for reconstructing the at least one second computed tomography dataset CD2 and/or for recording the at least one further two-dimensional X-ray projection image PB from a region to be mapped of an examination object 31 arranged on a patient positioning facility 32, the processing unit 22 may send a signal 24 to the X-ray source 33. Then, the X-ray source 33 may emit an X-ray beam, for example a cone beam and/or fan beam. When the X-ray beam arrives at a surface of the detector unit 34 after interaction with the region to be mapped of the examination object 31, the detector unit 34 may send a signal 21 to the processing unit 22. The processing unit 22 may, for example using the signal 21, create the plurality of two-dimensional X-ray projection images 1 for reconstructing the first computed tomography dataset CD1 and/or the plurality of two-dimensional X-ray projection images 2 for reconstructing the at least one second computed tomography dataset CD2 and/or the at least one further two-dimensional X-ray projection image PB. The processing unit 22 may then carry out an embodiment of the method for providing an evaluation dataset from a first medical three-dimensional computed tomography dataset CD1. The evaluation dataset BD1 may be created. The plurality of two-dimensional X-ray projection images 1 for reconstructing the first computed tomography dataset CD1 and/or the plurality of two-dimensional X-ray projection images 2 for reconstructing the at least one second computed tomography dataset CD2 may for example be received by the medical C-arm X-ray device 37 by an interface of the processing unit 22, for example by a computer-readable storage medium and/or a database.

In addition, the medical C-arm X-ray device 37 may include an input unit 41, for example a keyboard, and/or a depiction unit 42, for example a monitor and/or display. The input unit 41 may preferably be integrated in the depiction unit 42, for example in the case of a capacitive input display. An operator input on the input unit 41 may provide the method and/or the medical C-arm X-ray device 37 to be controlled.

The schematic depictions in the figures described do not represent any scale or size ratio.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for providing an evaluation dataset from a first medical three-dimensional computed tomography dataset, the method comprising:
reconstructing the first three-dimensional computed tomography dataset from a plurality of two-dimensional X-ray projection images recorded with different acquisition geometries from an examination object by a medical X-ray device;
applying a method for reducing artifacts to the first three-dimensional computed tomography dataset to generate an artifact-reduced image dataset;
identifying one or more stroke indications from the artifact-reduced image dataset;
creating an evaluation dataset by applying a method for evaluating a manifestation of the identified stroke indications to the artifact-reduced image dataset; and
providing the evaluation dataset.

2. The method as claimed in claim 1, further comprising:
creating a three-dimensional thrombus image from the artifact-reduced image dataset, the evaluation dataset, or the artifact-reduced image dataset or the evaluation dataset; and
providing the three-dimensional thrombus image;
wherein a thrombus is identified as a stroke symptom of the one or more stroke indications;
wherein the creation of the three-dimensional thrombus image includes segmentation of the thrombus from the artifact-reduced image dataset, the evaluation dataset, or the artifact-reduced image dataset and the evaluation dataset.

3. The method of claim 1, wherein the method for reducing artifacts includes at least one of registering the two-dimensional X-ray projection images to one another, applying a filter for noise reduction, or applying a filter for motion correction.

4. The method of claim 1, wherein the method for reducing artifacts comprises:
creating a three-dimensional auxiliary dataset by undersampling the first three-dimensional computed tomography dataset;
determining a slice along each of the two spatial directions of the three-dimensional auxiliary dataset, wherein each of the two slices includes a predetermined column and at least one line;
determining at least one rotation parameter, at least one translation parameter for reducing a line-by-line deviation with respect to the predetermined column, or at least one rotation parameter and at least one translation parameter for reducing a line-by-line deviation with respect to the predetermined column for each of the two slices; and
correcting the first three-dimensional computed tomography dataset by applying the at least one rotation parameter, the at least one translation parameter, or the at least one rotation parameter and the at least one translation parameter.

5. The method of claim 1, wherein identifying stroke indications is based on artificial intelligence.

6. The method of claim 1, wherein creating the evaluation dataset is based on artificial intelligence.

7. The method as claimed in claim 6, wherein a thrombus is identified as a stroke symptom, wherein input data of the method for evaluating the identified stroke indications is based on the artifact-reduced image dataset and at least one tissue parameter of the thrombus, wherein the at least one tissue parameter of the thrombus is determined independently of the first three-dimensional computed tomography dataset.

8. The method of claim 1, wherein output data of the manifestation of the identified stroke indications includes a prognosis, a workflow note, or the prognosis and the workflow note.

9. The method of claim 1, further comprising:
reconstructing at least one second three-dimensional computed tomography dataset, wherein the at least one second three-dimensional computed tomography dataset is reconstructed from a plurality of two-dimensional X-ray projection images recorded with different acquisition geometries from the examination object by the medical X-ray device;
providing at least one further artifact-reduced image dataset using the at least one second three-dimensional computed tomography dataset, wherein providing includes applying a further method for reducing artifacts;
creating a three-dimensional vessel image, wherein creating includes segmentation of vessels from the at least one further artifact-reduced image data set;
identifying vascular occlusions by applying a method for identifying vascular occlusions to the three-dimensional vessel image;
creating a plurality of maximum intensity projection images using the three-dimensional vessel image;
creating a further evaluation dataset by applying a further method for evaluating a manifestation of the identified vascular occlusions to the maximum intensity projection images; and
providing the further evaluation dataset.

10. The method of claim 9, wherein the further method for reducing artifacts comprises:
creating at least one further three-dimensional auxiliary dataset by undersampling the at least one second three-dimensional computed tomography dataset;
determining in each case a slice along two spatial directions of the at least one further three-dimensional auxiliary dataset, wherein each of the two slices includes a predetermined column and at least one line;
determining at least one rotation parameter, at least one translation parameter, or at least one rotation parameter and at least one translation parameter for reducing a line-by-line deviation with respect to the predetermined column for each of the two slices;
correcting the at least one second three-dimensional computed tomography dataset by applying the at least one rotation parameter, the at least one translation parameter, or the at least one rotation parameter and the at least one translation parameter.

11. The method of claim 9, further comprising:
recording at least one further two-dimensional X-ray projection image from the examination object by the medical X-ray device;
determining a common slice in the first three-dimensional computed tomography dataset and the second three-dimensional computed tomography dataset, wherein the common slice extends perpendicularly to the projection direction of the at least one further two-dimensional X-ray projection image;
creating a two-dimensional vessel slice image from the three-dimensional vessel image along the common slice;
creating a two-dimensional thrombus slice image from the three-dimensional thrombus image along the common slice;
creating a two-dimensional superimposed image;
wherein the creation of the two-dimensional superimposed image includes superimposition of the at least one further X-ray projection image and the two-dimensional vessel slice image, the two-dimensional thrombus slice image, or the two-dimensional vessel slice image and the two-dimensional thrombus slice image; and
providing the two-dimensional superimposed image.

12. The method of claim 11, wherein the determination of the common slice in the first three-dimensional computed tomography dataset and the second three-dimensional computed tomography dataset includes registration, wherein the first three-dimensional computed tomography dataset and the second three-dimensional computed tomography dataset are registered to one another, to the at least one further two-dimensional X-ray projection image, or to one another and to the at least one further two-dimensional X-ray projection image.

13. The method of claim 9, wherein the further method for reducing artifacts includes at least one of registering the two-dimensional X-ray projection images to one another, applying a filter for noise reduction, applying a filter for motion correction, or applying a filter for noise reduction and motion correction.

14. The method of claim 9, wherein a plurality of second three-dimensional computed tomography datasets are recorded in temporal succession; and wherein a phase of a perfusion is assigned to each of the second three-dimensional computed tomography datasets.

15. The method of claim 14, wherein the creation of the three-dimensional vessel image includes the assignment of an image value to each of the vessels in the three-dimensional vessel image, wherein the image values are determined by the phases of the plurality of second three-dimensional computed tomography datasets.

16. The method of claim 14, wherein the further method for reducing artifacts includes creation of at least one three-dimensional difference image data set;
wherein, to create the difference image data set, a three-dimensional mask image is subtracted from at least one of the plurality of second three-dimensional computed tomography datasets.

17. The method of claim 9, wherein the identification of the vascular occlusions in the three-dimensional vessel image is based on artificial intelligence.

18. The method of claim 9, wherein the creation of the further evaluation dataset is based on artificial intelligence.

19. A system for providing an evaluation dataset from a first medical three-dimensional computed tomography dataset, the system comprising:
a medical x-ray device configured to record a plurality of two-dimensional X-ray projection images with different acquisition geometries of an examination object; and
a processing unit configured to reconstruct the first three-dimensional computed tomography dataset from the plurality of two-dimensional X-ray projection images, apply a method for reducing artifacts to the first three-dimensional computed tomography dataset to generate an artifact-reduced image dataset, identify one or more stroke indications from the artifact-reduced image dataset, create an evaluation dataset by applying a method for evaluating a manifestation of the identified stroke indications to the artifact-reduced image dataset, and provide the evaluation dataset.

20. A non-transitory computer implemented storage medium that stores machine-readable instructions executable by at least one processor for providing an evaluation dataset from a first medical three-dimensional computed tomography dataset, the machine-readable instructions comprising:
providing an evaluation dataset from a first medical three-dimensional computed tomography dataset;
reconstructing the first three-dimensional computed tomography dataset from a plurality of two-dimensional X-ray projection images recorded with different acquisition geometries from an examination object by a medical X-ray device;
applying a method for reducing artifacts to the first three-dimensional computed tomography dataset to generate an artifact-reduced image dataset;
identifying one or more stroke indications from the artifact-reduced image dataset;
creating an evaluation dataset by applying a method for evaluating a manifestation of the identified stroke indications to the artifact-reduced image dataset; and
providing the evaluation dataset.
